(19) United States
(12) Patent Application Publication
(10) Pub. No.: US 2012/0114724 A1
(43) Pub. Date: May 10, 2012

(54) ANTIMICROBIAL AGENT, METHOD OF PREPARING AN ANTIMICROBIAL AGENT AND ARTICLES COMPRISING THE SAME

(75) Inventors: Ronald Howard Baney, Gainesville, FL (US); Samuel Ralph Farrah, Fort Worth, TX (US); Le Song, Quakertown, PA (US)

(73) Assignee: UNIVERSITY OF FLORIDA RESEARCH FOUNDATION, INC., Gainesville, FL (US)

(21) Appl. No.: 12/941,687

(22) Filed: Nov. 8, 2010

(51) Int. Cl. A01N 25/34; A01N 25/22; B05D 5/00; B02C 19/06; A61L 2/18; A01N 43/04; A01N 43/16

(52) U.S. Cl. 424/411; 514/54; 424/126; 514/57; 514/60; 514/55; 514/59; 514/56; 241/5; 427/2.1; 427/244

(57) ABSTRACT

The present disclosure is directed to method of preparing an antimicrobial agent comprising heating a dialdehyde polysaccharide. The method comprises subjecting a dialdehyde polysaccharide, such as a dialdehyde starch or a dialdehyde cellulose, to heating and/or sonication for a period of time. Also provided herein is an antimicrobial composition comprising the prepared dialdehyde polysaccharide. The antimicrobial composition is effective at killing microbial agents such as viruses and bacteria within a short period of time.

### CROSS REFERENCE TO RELATED APPLICATIONS

This application claims priority to U.S. Patent Application No. 61/051,860, filed on May 9, 2008, and to PCT/US09/043456 filed on May 11, 2009, the content of which in its entirety is herein incorporated by reference.

### BACKGROUND

Several major, and potentially catastrophic challenges, are presently confronting health officials around the world. One of these potential challenges lies in avian influenza, also known as “bird flu”. While the occurrence of bird flu in human individuals has been documented, to date the occurrence of cases in human has been both sporadic, and limited. At present, the transmission of avian influenza from one individual to another has been limited to the avian species. However, experts predict that if avian influenza were ever to cross over to the human species, and become transmitted from human to human, a pandemic of epic proportions could potentially result. Current strategies for the control of a potential infection call for the vaccination of a limited number of first responders, and the quarantine and isolation of a community population for up to two weeks. Surgical masks are also considered to be a backup strategy for controlling the transmission rate, since they have the ability to partially remove airborne particles generated by a cough or a sneeze. However, these masks are lacking in their ability to kill any viruses that come in contact with the mask, and as such, viral particles retain their ability to travel into, and subsequently infect, the lungs. Further, it is reasonable to assume, that because individuals in our society are so interdependent on one another for food, energy, and transportation, that quarantine strategies would not be effective methods for controlling the spread of the infectious disease.

A second major concern in developing a strategy for curtailing the spread of such disease is the development, through mutation, of “superbugs”, bacterial strains which are resistant to antibiotics currently available on the market. Among these, infections due to multi-drug resistant tuberculosis (MDR-TB) and drug-resistant strains of Staphylococcus aureus, are rapidly becoming prevalent in places where close physical contact between individuals is not only possible, but highly probable. Included in these are hospitals, nursing homes, prisons, and sports organizations. In fact, it is estimated that up to 5% of all nursing homes are presently harboring the various strains of drug-resistant bacteria.

Dialdehyde polysacchardides are polymeric dialdehydes prepared by the selective oxidation of polysaccharides through the use of periodate salts. Due to the presence of dialdehyde functional groups in the polymer chain, dialdehyde polysaccharides have the ability to react with hydroxyl, amino, imino and sulfhydryl functional groups. One type of known dialdehyde polysaccharide is dialdehyde starch (DAS). The application of DAS to a variety of different and diverse fields has been investigated including paper, leather and textile applications, as well as biomedical applications, for example, the surface modification of stents to improve protein absorption. The toxicity of DAS has also been determined in rats and is reported to have an extremely low oral acute toxicity in rats, i.e. an acute LD50 for a 10% DAS aqueous suspension is greater than or equal to 6800 mg/kg (Radley, J. A., Starch Production Technology. 1976, Applied Science Publishers: London).

While the application of dialdehyde polysaccharides as antimicrobial agents has been investigated to some degree, research into the antimicrobial behavior of dialdehyde polysaccharides has not been fully explored. U.S. Pat. No. 4,034,084 to Serigusa, describes the antimicrobial activity of dialdehyde cellulose granules on select bacterial strains, and discloses that these insoluble granules were able to inhibit the growth of a select number of bacterial strains.

A new suspension has been discovered for producing a new low cost, viral filtering and viricidal mask, and for a new strategy for addressing the challenge created by antibiotic-resistant bacteria.

### SUMMARY

Disclosed herein is a method of preparing an antimicrobial agent, comprising heating a dialdehyde starch in water at a temperature of about 60° C. to about 120° C. to form a dispersion of dialdehyde polysaccharide in water.

Disclosed herein too is an antimicrobial composition, comprising a dispersion of a dialdehyde starch in water having a pH of about 2.5 to about 9.

Disclosed herein too is a method of inhibiting the growth of a microbial agent, the method comprising contacting the microbial agent with a composition comprising a dispersion of dialdehyde starch in water; the dispersion having a pH of about 2.5 to about 9.

Disclosed herein too is a method of producing an antimicrobial article comprising heating a dialdehyde starch in water at a temperature of about 60° C. to about 120° C. to form a dispersion of dialdehyde starch in water; sonicating the dispersion of dialdehyde starch in water; and contacting the article with the dispersion of dialdehyde starch in water to form an antimicrobial article.

Disclosed herein too is a composition comprising a dialdehyde polysaccharide; and water; the dialdehyde polysaccharide being dispersed in the water; the dialdehyde polysaccharides having average particle sizes of about 5 to about 150 nanometers.

Disclosed herein too is an article comprising the aforementioned composition.

Disclosed herein too is a filter comprising a substrate; and a dialdehyde polysaccharide disposed upon the substrate.

Disclosed herein too is a method comprising oxidizing a cellulose; forming a dialdehyde polysaccharide on a surface of the cellulose; and using the cellulose having the dialdehyde polysaccharide disposed thereon as a filter.

### BRIEF DESCRIPTION OF THE DRAWINGS

FIG. 1 is a graph illustrating the effect of pH on the log reduction of Gram-negative bacteria following bacterial incubation in PBS at varying pH levels for a period of one hour;

FIG. 2 is a graph illustrating the effect of dialdehyde starch on the log reduction of Gram-negative bacteria following bacterial incubation in dialdehyde starch of varying pH levels for a period of one hour;

FIG. 3 is a graph illustrating the effect of pH on the log reduction of Gram-positive bacteria following bacterial incubation in PBS of varying pH levels for a period of one hour;

FIG. 4 is a graph illustrating the effect of dialdehyde starch on the log reduction of Gram-positive bacteria following bacterial incubation in dialdehyde starch of varying pH levels for a period of one hour;

FIG. 5 is a bar graph showing the log reduction in Gram-negative and Gram-positive bacteria observed following bacterial treatment with either dialdehyde starch or PBS, each at a pH of 4.8, for one or four hours;

FIG. 6 is a graph illustrating the effect of DAS sonication on the inactivation of bacteria;

FIG. 7 is a graph comparing the effect of either 2.7% dialdehyde starch or PBS having the same pH value, on the log reduction of PRD1 virus following treatment for 1 hour or 4 hours;

FIG. 8 is a graph comparing the effect of either 2.7% dialdehyde starch or PBS having the same pH value, on the log reduction of MSD1 virus following treatment for 1 hour or 4 hours;

FIG. 9 is a graph comparing the effect of either 2.7% D dialdehyde starch or PBS having the same pH value, on the log reduction of polio virus following treatment for 1 hour or 4 hours;

FIG. 10 depicts structural differences between the dialdehyde starch and the oxidized corn starch;

FIG. 11(a) is a gel permeation chromatography graph showing the response versus the retention time, while the FIG. 11(b) is another gel permeation chromatography graph showing the differential weight fraction versus molecular weight;

FIG. 12 is a graph of absorbance versus wavelength for a dialdehyde starch aqueous suspension; the graph is a spectrum obtained by Fourier Transform Infrared analysis;

FIG. 13 is an Ultraviolet-Visible (UV-Vis) spectrum. FIG. 13(a) is a UV-Vis spectra of the dialdehyde starch samples taken in the reflectance mode for as-received dialdehyde starch. FIG. 13(b) is a UV-Vis spectra of the freeze-dried sample of the 3% as-prepared dialdehyde starch aqueous supernatant taken in the reflectance mode. FIG. 13(c) is a UV-Vis spectra taken in the transmission mode of the 3% as-prepared DAS aqueous supernatant at pH=3, diluted 100 times using same pH PBS buffer. FIG. 13(d) is a UV-Vis spectra taken in the transmission mode of the 0.3% DAS granular suspension; and

FIG. 14 is a schematic diagram of the setup used to measure the efficacy of the dialdehyde polysaccharides as filters.

### DETAILED DESCRIPTION

The terms “a” and “an” as used herein do not denote a limitation of quantity, but rather denote the presence of at least one of the referenced item. All ranges disclosed herein are inclusive and combinable.

The terms “comprises” and/or “comprising,” as used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third, and the like, may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The term “comprising” as used herein may be substituted by “consisting of or “consisting essentially of”. In addition, the use of the term “about” preceding a numeral is intended to include that numeral. For example, the use of the phrase “about 0.1 to about 1” is intended to mean that both 0.1 and 1 are included in the range. In addition, all numbers and ranges disclosed herein are interchangeable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

As used herein, the term “microbial agent” refers to a microorganism, such as a virus or bacteria. The microbial agent may, or may not, be capable of causing morbidity and/or mortality in either humans or animals. As used herein, an “antimicrobial agent” is an agent that has antiviral (kills or suppresses the replication of viruses), or antibacterial (bacteriostatic or bactericidal) properties.

“Polysaccharides” as used herein, are biological polymers made up of repeating monosaccharides joined together by glycosidic bonds, and are large, often branched, macromolecules. In biological systems, polysaccharides function as structural components or as energy storage molecules.

The present disclosure is directed to a method of preparing an antimicrobial agent having both antiviral and antibacterial properties. The antimicrobial agent prepared as described herein, has the ability to effectively kill Gram-negative and Gram-positive bacteria, as well as bacterial and human viruses, within a period of less than or equal to about 4 hours.

The present disclosure is also directed to compositions that comprise the antimicrobial agent. These antimicrobial agents can be applied to surfaces that can then be used to effectively kill Gram-negative and Gram-positive bacteria, as well as bacterial and human viruses.

This disclosure also relates to articles comprising the antimicrobial agent. An exemplary article is a filter having a layer of dialdehyde polysaccharides disposed thereon. In one embodiment, the layer of dialdehyde polysaccharides may be physically extractable from a substrate upon which is it is disposed. In another embodiment, the layer of dialdehyde polysaccharides may be covalently bonded with the substrate upon which it is disposed and may not be physically separated from the substrate without undergoing some form of physical, chemical or thermal degradation.

In one embodiment, the antimicrobial agent is a dialdehyde polysaccharide that has been subjected to heating and/or sonication in water to produce a dispersion of a dialdehyde polysaccharide in water. In one embodiment, the dispersion of the dialdehyde polysaccharide in water can have the consistency of a gel-like material.

The source of the polysaccharide used to prepare the dialdehyde polysaccharide may be from corn, wheat, potato or tapioca starches, celluloses, dextrins, dextrans, algins, insulins and related materials. Specifically, the dialdehyde polysaccharide is prepared from a starch or a cellulose.

In one embodiment, the antimicrobial agent is a dialdehyde polysaccharide selected from the group consisting of a dialdehyde starch (DAS), a dialdehyde cellulose, cellulose, alkyl cellulose, e.g., methyl cellulose, hydroxyalkyl cellulose, alkylhydroxyalkyl cellulose, cellulose sulfate, salts of carboxymethyl cellulose, carboxymethyl cellulose, carboxyethyl cellulose, chitin, carboxymethyl chitin, hyaluronic acid, salts of hyaluronic acid, alginate, alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, chitosan, heparin, heparin sulfate, heparin sulfate, dermatan sulfate, keratin sulfate, carrageenans, chitosan, starch, amylose, amylopectin, poly-N-glucosamine, polymannuronic acid, polyglucuronic acid, polyguluronic acid, and derivatives of any of the above, or a combination comprising one or more of the foregoing dialdehyde polysaccharides.

In one exemplary embodiment, the polysaccharide is oxidized as described herein to assure that the aldehyde-modified polysaccharide is biodegradable. In an exemplary embodiment, the dialdehyde polysaccharide is a dialdehyde starch that has been heated in water and/or sonicated. A portion of the dialdehyde starch solubilizes in the water while a portion remains undispersed. In one embodiment, the undispersed portion can be filtered leaving behind a suspension of the dialdehyde starch in water.

In another exemplary embodiment, the dialdehyde polysaccharide is essentially free of functional or reactive moieties other than aldehyde moieties. By essentially free, it is meant that the polysaccharide does not contain such functional or reactive moieties in amounts effective to alter the properties of the dialdehyde polysaccharide.

Starch, is a polysaccharide comprising a mixture of two complex carbohydrate polymers, amylose and amylopectin. Both amylose and amylopectin are polymers of D-glucose units bonded together via alpha-linkages. In amylose, the glucose units are linked via α-1, 4 linkages with the ring oxygen atoms all on the same side, whereas in amylopectin about one glucose unit in every twenty or so repeat units is also linked via an α-1,6 linkage, thereby forming branch-points. Thus, amylose comprises a linear chain of several hundred glucose molecules, while amylopectin is a branched molecule made of several thousand glucose units. The ratio of amylose to amylopectin in starch is generally from about 20 to about 30 mole percent amylose, to about 70 to about 80 mole percent amylopectin. The relative proportions of amylose to amylopectin, and the branch-points, both depend on the source of the starch. For example, amylomaizes contain over 50% mole percent amylose whereas “waxy” maize has almost none (˜3 mole percent).

Cellulose is a structural polysaccharide founds in plants, comprising a linear chain of D-glucose. The glucose units of cellulose are bonded together via beta-linkages and are held together by intra and inter chain hydrogen bonds. Like starch, cellulose is also insoluble in water. The structure of cellulose is generally more crystalline than the structure of starch.

The preparation of dialdehyde polysaccharides such as dialdehyde starch or dialdehyde cellulose generally occurs by the selective oxidation of the polysaccharide polymer. A variety of oxidizers can be used to oxidize the polysaccharide. Examples of oxidizers include those selected from the group consisting of alkali, alkaline earth and transition metal salts of, for example, periodate, hypochlorite, perbromate, chlorite, chlorate, hydrogen peroxide, peracetic acid and combinations comprising at least one of the foregoing oxidizers.

Specifically, the oxidation of a polysaccharide is conducted using periodate salts as the oxidizing agent. Periodic acid is an oxidizing agent that breaks the C—C bond between two adjacent hydroxyl groups. Periodates such as for example sodium periodate, potassium periodate, and the like can also be used as oxidizing agents.

Through this process, the 1,2-diol group of glucose is converted into a dialdehyde. A reaction depicting the preparation of a dialdehyde polysaccharide by the selective oxidation of starch, is shown in Equation I:

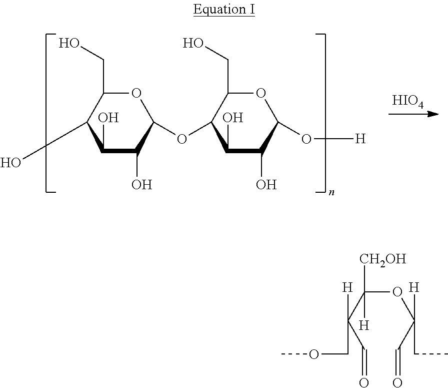

As shown in Equation 1, the oxidation of starch, results in the addition of two aldehyde groups to individual glucose molecules within the polymer chain. The advantage of using periodic acid lies in the specificity of its oxidation. It facilitates the formation of aldehydes within the polysaccharide molecule.

The extent of oxidation of the polysaccharide polymer can be controlled by, for example, the amount of oxidizer added, the duration of the oxidation process, and/or the temperature of the reaction. For example, the oxidation time needed for the oxidation of starch, can be attained in about 24 hours. Specifically, at least about 15 percent of the hydroxyl groups are oxidized, and more specifically, about 35 to about 100 percent of the hydroxyl groups are oxidized.

In one embodiment, a method of preparing an antimicrobial agent comprises suspending granules of the dialdehyde polysaccharide (e.g., a dialdehyde starch) in water to form an aqueous dispersion, and heating the dialdehyde polysaccharide for a period of time effective to increase the antimicrobial activity of the dialdehyde polysaccharide. Prior to heating, the dialdehyde polysaccharide is highly granular and insoluble in water. Upon heating, the dialdehyde polysaccharide loses its insoluble nature, resulting in the solubilization of the dialdehyde polysaccharide and the formation of a dispersion of dialdehyde polysaccharide in water having the consistency of a gel-like material.

In another embodiment, the dispersion of dialdehyde polysaccharide in water comprises about 0.2 to about 40 weight percent dialdehyde polysaccharide, specifically about 1 to about 30 weight percent, and more specifically about 2 to about 20 weight percent, based on the total weight of the dialdehyde polysaccharide and water. An exemplary amount of dialdehyde polysaccharide in the dispersion of dialdehyde polysaccharide in water is about 3 to about 10 weight percent, based on the total weight of the dialdehyde polysaccharide and water. As noted above, an exemplary dialdehyde polysaccharide for dispersion in water is a dialdehyde starch.

In one embodiment, the viscosity of a dispersion of the dialdehyde polysaccharide in water is about 0.03 to about 0.3 poise, specifically about 0.05 to about 0.2 poise, and more specifically about 0.07 to about 0.1 poise.

In another embodiment, the average particle sizes of the dialdehyde polysaccharide is about 5 nanometers to about 150 nanometers, specifically about 7 to about 100 nanometers, and more specifically about 8 to about 75 nanometers.

In the case of dialdehyde starch, the heating of the dialdehyde starch in water is thought to cause both the swelling of the starch granule and ultimately the loss of granular integrity. The swelling and/or breakdown of the dialdehyde starch molecule results in an overall increase in the number of exposed reactive dialdehydes thereby providing additional reactive groups that are capable of interacting with the microbial agent.

In one embodiment, the heating of the dialdehyde starch increases the percentage of dialdehyde reactive groups that are exposed in the dispersion of the dialdehyde starch in water as compared to the number of dialdehyde reactive groups exposed on the granular dialdehyde polysaccharide prior to heating. The percentage of reactive dialdehyde groups present in the dispersion of the dialdehyde starch is increased by about 10 to about 50 percent, as compared with the number of reactive dialdehyde groups present on the surface of a granular dialdehyde polysaccharide.

In one embodiment, the method of preparing the antimicrobial agent comprises heating the dialdehyde starch in water for a period of about 1 to about 4 hours at a temperature of about 80° C. to about 120° C. Pressures greater than or equal to atmospheric can be used during the heating.

Specifically, the dialdehyde starch is heated for a period of about 1.5 to about 3 hours and more specifically, for a period of about 2 to about 2.5 hours. The heating of the sample may be conducted using known methods for heating. The dialdehyde starch may be mixed during the heating process to ensure the even distribution of heat throughout the suspension. Methods of heating of the dialdehyde starch may be selected from convection, conduction, radiation, or a combination comprising at least one of the foregoing heating methods.

In another embodiment, the method of preparing the antimicrobial agent comprises sonicating the dispersion of the dialdehyde starch in water. Prior to or during sonication, the dialdehyde starch may first be heated using the methods described herein. Alternatively, the dialdehyde starch may be left untreated i.e., not heated prior to sonication. Another possible alternative comprises first sonicating the dialdehyde starch and then heating the dialdehyde starch as previously described. Yet another alternative comprises simultaneously sonicating and heating the dialdehyde starch.

In one embodiment, the dialdehyde starch is subjected to sonication for a period of about 10 minutes to about 3 hours. Specifically, the dialdehyde starch is sonicated for a period of about 15 minutes to about 90 minutes. More specifically, the dialdehyde starch is sonicated for about 30 minutes to about 60 minutes. The sonication process may be conducted in a continuous manner for a shorter period of time, or in an intermittent manner, for a longer period of time. Sonication can be conducted at a power of up to about 10 watts to promote the dispersion of the dialdehyde starch in the water.

The sonication of the dialdehyde starch breaks down the dialdehyde polysaccharide granules into smaller particles. The breakdown of the dialdehyde polysaccharide and the generation of smaller particles, results in an overall increase in the surface area. In turn, the increased surface area provides for the exposure of more dialdehyde groups thereby providing additional reactive groups that are capable of interacting with the microbial agent. Prior to sonication, the dialdehyde polysaccharide granules, or particles range in size from about 0.01 micrometers (μm) to about 10 millimeters. Following sonication, the size of the dialdehyde particles is about 5 nanometers to about 500 μm. In addition, sonication of the particles subsequent to heating acts to further reduce the size of the particles and therefore further increases the surface area available for interaction with the microbial agent(s).

In one embodiment, a portion of dialdehyde polysaccharide granules upon being dispersed into the water are completely solubilized in the water, while a portion may remain in the form of particles.

In another embodiment, up to about 99 weight percent of the dialdehyde polysaccharides may be dispersed in the water during the cooking and/or sonication. In yet another embodiment, up to about 90 weight percent of the dialdehyde polysaccharides may be dispersed in the water during the cooking and/or sonication. In yet another embodiment, up to about 80 weight percent of the dialdehyde polysaccharides may be dispersed in the water during the cooking and/or sonication.

In yet another embodiment, about 1 to about 70 weight percent of the dialdehyde polysaccharides may be dispersed in the water during the cooking and/or sonication. The weight percents are based on the sum of the weights of the dialdehyde polysaccharides and water. The remaining portion of the dialdehyde polysaccharides may be retained in the form of particles.

In one embodiment, the particle size of dialdehyde polysaccharide particles after dispersion in water can be about 0.5 nanometers to about 500 micrometers, specifically about 20 nanometers to about 250 micrometers, and more specifically about 50 nanometers to about 100 micrometers. Undispersed dialdehyde polysaccharide particles (particles that are large in size that are larger than about 500 micrometers) can be separated from the suspension and can be removed. The separation can be effected by filtration, centrifugation, decantation, and the like.

As a result of filtration, the antimicrobial agent may comprise up to about 99 weight percent of the dialdehyde starch dispersed in the water. In one embodiment, as a result of the filtration, the antimicrobial suspension can comprise about 1 to about 90 weight percent, specifically about 3 to about 60 weight percent and more specifically about 5 to about 40 weight percent of the dialdehyde starch that is dispersed in water.

As noted above, it is desirable to disperse dialdehyde starch in water upon heating. In an exemplary embodiment, particle size analysis measured on a Coulter Counter of: 1) a 3% dialdehyde starch “granular” suspension and 2) a dialdehyde starch-aqueous suspension, (“cooked”) was almost the same with mean particle sizes of about 0.5 micrometers to about 2 micrometers, specifically 0.7 to about 1.8 micrometers, and more specifically about 0.9 to about 1.5 micrometers. In an exemplary embodiment, the mean particle sizes were about 1.3 micrometers. After centrifuging of 1) and 2), the suspensions both had separated into two phases, an aqueous phase and solid pellet phase. The two aqueous phases were freeze dried and the remaining residues were then weighted. There was no measurable solid in the aqueous phase of the dialdehyde starch “granular” suspension 1) for the “cooked” sample. For the sample 2), 94% of the sample was water-soluble. Particle size analysis of the aqueous phase of the dialdehyde starch-aqueous suspension, after centrifuging indicated it that the size had significantly dropped to the mean size of 62 nm.

In one embodiment, a method is provided for inhibiting the growth of a microbial agent by contacting the microbial agent with a composition comprising a dialdehyde polysaccharide that has been heated according to the method described herein.

In another embodiment, a method is provided for inhibiting the growth of a microbial agent by contacting the microbial agent with a composition comprising a dialdehyde polysaccharide that has been sonicated.

The heating and/or sonication of the dispersion of dialdehyde polysaccharide in water, is effective to increase both the antibacterial and antiviral activity of the dialdehyde polysaccharide. Specifically, the dialdehyde polysaccharide prepared by the method described herein, is able to effectively kill both bacteria and viruses. Specifically, a sterilizing preparation capable of killing viruses and bacteria comprising from about 0.05 to about 5.0 weight percent (wt %) of a dispersion of dialdehyde polysaccharide is capable of killing bacteria and viruses. More specifically, liquid or gel preparations comprising, about 2 to about 3 wt % of a dispersion of active dialdehyde polysaccharide is capable of killing bacteria and viruses.

A method of inhibiting the growth of a microbial agent using a heated and/or sonicated dialdehyde polysaccharide comprises contacting the dispersion of dialdehyde polysaccharide with the bacteria or virus for a period of time effective to inactivate or kill the bacteria or virus. Specifically, the dialdehyde polysaccharide is effective at killing the microbial agent. As used herein, the terms “killing” or “inhibition” are used interchangeably, and are indicative of the absence of microbial growth and/or replication following contact with the antimicrobial dialdehye polysaccharide.

The dialdehyde polysaccharide has two closely spaced aldehyde groups (dialdehyde) capable of reacting with hydroxyl, amino, imino, and sulfhydryl groups. As a result, the dispersion of dialdehyde polysaccharide is highly effective at cross-linking both microbial proteins as well as microbial nucleic acid. The cross-linking of cellular proteins results in the antimicrobial action of the compound, either by causing a release of microbial cell content into the surrounding medium, and/or by interacting with the cell wall of the microbial agent, thereby interfering with the metabolic processes of the organism and causing the killing action.

Both the pH of the dispersion of dialdehyde polysaccharide, and the time that the microbial agent is in contact with the dispersion of dialdehyde polysaccharide, impact the level of killing of the microbial agent. A composition comprising the dispersion of dialdehyde polysaccharide is effectively able to inhibit the growth of the microbial agent across a wide range of pH. Specifically, the dialdehyde polysaccharide is effective in killing bacteria and viruses within a pH of about 2.5 to about 9. However, at lower pH levels, that is, at a more acidic pH, the period of time effective to kill the microbial agent is shorter than the amount of time effective for a dispersion of dialdehyde polysaccharide at a higher pH. That is, at a more neutral or basic pH, the amount of time effective to kill the microbial agent is increased.

In one embodiment, the dialdehyde polysaccharide is effective in killing bacteria and viruses within a pH of about 3 to about 8. In another embodiment, the dialdehyde polysaccharide is effective in killing bacteria and viruses within a pH of about 4 to about 6.

The period of time effective for the dialdehyde polysaccharide to kill the bacteria or virus ranges from about 0.5 hours to about 10 hours, specifically, from about 1 hour to about 4 hours. At an acidic pH, the period of time effective to kill the bacteria or virus ranges from about 0.5 to about 2 hours, specifically, about 1 hour. At a more neutral or basic pH, the period of time effective to kill the microbial agent is about 3 to about 10 hours, specifically, about 4 hours.

The antimicrobial agent comprising a heated and/or sonicated dialdehyde polysaccharide as described herein, has the ability to inactivate a wide variety of microbial agents. Specifically, the antimicrobial agent is particularly effective against microbial agents that cause morbidity and/or mortality in both humans and animals. The microbial agents may be readily transmitted between individuals of the same, and/or different species, or may be opportunistic pathogens, that is, bacterial or viral strains that exploit some break in the host defenses to initiate an infection. Examples of microbial agents include viruses, for example single stranded, and double stranded RNA or DNA viruses; Gram-positive bacteria, and Gram-negative bacteria.

Examples of viruses capable of causing morbidity and/or mortality include those selected from the group consisting of influenza virus; encephalitis causing viruses, for example, Eastern and Western equine encephalitis; hemorrhagic fever-causing viruses, for example, Lassa fever, Dengue fever, Ebola virus, and Hantavirus; polioviruses; severe acute respiratory syndrome (SARS)-associated coronavirus; and Hepatitis C. Other examples of viruses that can be inactivated by the dispersion of dialdehyde polysaccharide include those that give rise to the common cold such as for example, over 100 serotypes of rhinoviruses (a type of picornavirus), coronavirus, human parainfluenza viruses, human respiratory syncytial virus, adenoviruses, enteroviruses, or metapneumovirus.

Examples of bacterial strains capable of causing morbidity and/or mortality, include those selected from the group consisting of drug-resistant Staphylococcus aureus, multi-drug resistant Mycobacterium tuberculosis, Escherichia coli, Salmonella species, for example Salmonella typhi, Pseudomonas aeruginosa, Enterococcus faecalis, Bacillus cereus, Clostridium difficile, Helicobacter pylori, Streptococcus, Group A, Yersinia pestis, Vibrio cholerae, Francisella tularensis, Rickettsia rickettsii, Bacillus anthracis, Coxiella burnetii and Clostridium botulinum.

The antimicrobial agent can be used to disinfect surfaces or articles of manufacture thereby ensuring that the affected surface or article is free from contamination by bacteria and/or viruses. The antibacterial agent can also be used to prevent the contamination of surfaces, or articles of manufacture. A method of preserving, sanitizing, disinfecting or sterilizing a contaminated surface or area using a composition comprising the antimicrobial agent comprising an effective amount of dialdehyde polysaccharide, comprises the steps of contacting the composition with the contaminated surface or area for a period of time effective to preserve, sanitize, disinfect or sterilize the surface or area.

In one embodiment, in one method of disinfecting surfaces, the antimicrobial agent is sprayed onto a surface that is to be disinfected. The water from the antimicrobial agent is allowed to evaporate, leaving a film of dialdehyde polysaccharide (e.g., dialdehyde starch) on the surface. The film of dialdehyde polysaccharide can kill any bacteria or virus that come into contact with the surface. The antimicrobial agent can be used in the form of an aerosolized spray.

The film can have a thickness of about 10 nanometers to about 500 micrometers, specifically about 20 nanometers to about 250 micrometers, and more specifically about 30 nanometers to about 100 micrometers.

In another embodiment, the dialdehyde polysaccharide that is dispersed in water can be precipitated in the form of a fine powder. The precipitation can be brought about by freeze drying, or by the addition of a liquid (that is not compatible with the dialdehyde polysaccharide) to the suspension of dialdehyde polysaccharide in water. The powder can then be applied to parts of the body (e.g., the armpits, groins, and the like) or to other surfaces where disinfection is desired. Powder particles can have particle sizes of about 10 nanometers to about 200 micrometers.

The incorporation of one or more antimicrobial agents into an article or item of protective material provides an additional protection mechanism, acting to inactivate, or suppress the growth of microbial agents, such as bacteria, and viruses, that come into contact with the protective material. The antimicrobial agent can be used as a component in, or on the surface of, a variety of articles of manufacture, including articles of protective apparel, such as masks, gloves, clothing, garments or other items intended to protect the wearer or user against harm or injury as caused by exposure to microbial agents. The antimicrobial agent can also be used as a component of tampons, incontinence pads, sheets, and curtains.

The antibacterial agent may also be applied as a coating on the surface of medical devices. “Medical device” refers to any intravascular or extravascular medical devices, medical instruments, foreign bodies including implants and the like.

The term “medical device” also includes surgical or burn dressings, adhesive bandages or any external device that can be applied directed to the skin. Examples of intravascular medical devices and instruments include balloons or catheter tips adapted for insertion, prosthetic heart valves, sutures, surgical staples, synthetic vessel grafts, stents, stent grafts, vascular or non-vascular grafts, shunts, aneurysm fillers, intraluminal paving systems, guide wires, embolic agents, filters, drug pumps, arteriovenous shunts, artificial heart valves, artificial implants, foreign bodies introduced surgically into the blood vessels or at vascular or non-vascular sites, leads, pacemakers, implantable pulse generators, implantable cardiac defibrillators, cardioverter defibrillators, defibrillators, spinal stimulators, brain stimulators, sacral nerve stimulators, chemical sensors, breast implants, interventional cardiology devices, catheters, and the like. Examples of extravascular medical devices and instruments include plastic tubing, catheters, dialysis bags or membranes whose surfaces come in contact with the blood stream of a patient.

In one embodiment, one manner of proceeding provides a method of attaching a dialdehyde polysaccharide to a surface, the method comprising placing a dispersion of dialdehyde polysaccharide in water on the surface for a period of time sufficient for at least a portion of the dialdehyde polysaccharide to be adsorbed by the surface; and drying the surface at a temperature of from about 50° C. to about 150° C. The thus applied dialdehyde polysaccharide functions to kill both bacteria and viruses that come into contact with the surface.

In one embodiment, a composition comprising the antimicrobial agent may be used in a filter. The dialdehyde polysaccharides (that are obtained by the oxidation of the polysaccharides) and/or the dialdehyde starch in a suspension of water may be disposed on a porous substrate such as for example, a mesh, a gauze, a porous paper, a weave, a textile, or the like. The porous substrate may comprise a polymer, a metal, a ceramic, or a combination comprising a polymer, a metal or a ceramic. The porous substrate may be then be optionally dried to remove the moisture. The porous substrate with the dialdehyde polysaccharides and/or the dialdehyde starch disposed thereon may then be used as a filter.

In an exemplary embodiment, the porous substrate can comprise cellulose. In another embodiment, the porous substrate can comprise oxidized cellulose. Examples of different types of celluloses are provided above.

As noted above, the dialdehyde polysaccharides and/or the dialdehyde starch may be disposed upon a porous substrate. In one embodiment, a cellulose substrate may be subjected to oxidation to form a layer of dialdehyde polysaccharides on the surface. As noted above, polysaccharides can be oxidized with oxidizers; the oxidizers being alkalis, alkaline earth and transition metal salts of, for example, periodate, hypochlorite, perbromate, chlorite, chlorate, hydrogen peroxide, peracetic acid and combinations comprising at least one of the foregoing oxidizers.

The cellulose substrate may be exposed to the oxidizing agent for a period of time effective to form a layer of dialdehyde polysaccharide (as shown in equation I above) on the surface. The total amount of time for the oxidation may be about 10 minutes to about 5 hours. In one embodiment, a fibrous substrate comprising cellulose subjected to oxidation may subsequently be woven to form a weave or a textile that can be used as a filter.

Thus in one embodiment, a filter that comprises dialdehyde polysaccharides can comprise a plurality of layers that can be physically separated. The dialdehyde polysaccharide is generally the outermost layer of the filter that will be contacted by bacteria and viruses. On the other hand, a filter can comprise a layer of dialdehyde polysaccharides that is covalently bonded (reacted) with the substrate. In other words, the substrate and the layer form a single unitary indivisible structure. This structure cannot be physically separated without undergoing some form of mechanical, thermal and/or chemical degradation. In one embodiment, only a portion of the substrate can be converted to a dialdehyde polysaccharide upon oxidation. In another embodiment, the entire substrate can be converted to a dialdehyde polysaccharide upon oxidation.

The filter can comprise pores that are in the micrometer range or in the nanometer range. In one embodiment, the pores have an average pore size of about 50 to about 2,500 nanometers, specifically about 100 to about 1,500 nanometers, and more specifically about 150 to about 1,000 nanometers.

In one embodiment, a composition comprising the antimicrobial agent may also contain additives such as pigments, fragrances, anticorrosion agents, stabilizers such as triethylene glycol, and surfactants. Examples of surfactants include quaternary ammonium compounds, nonionic, and anionic surfactants. Quaternary ammonium compounds not only function as surfactants but aid in antimicrobial activity. Nonionic surfactants can provide increased stability to the antimicrobial composition. Examples of nonionic surfactants include those selected from the group consisting of water insoluble alcohols, for example, octanol, decanol, dodecanol, and the like; phenols, for example, octyl phenol, nonyl phenol, and the like; and ethoxylates of the above-mentioned alcohols and phenols, for example, ethoxylates having from about 1 to about 10 moles of ethylene oxide per mole of alcohol or phenol. Other nonionic surfactants that can be used include ethylene oxide/propylene oxide block copolymers. When surfactants are employed they can comprise about 0 to about 89 wt %, preferably about 1 to about 50 wt % of the composition.

The invention is further illustrated by the following, non-limiting examples.

### EXAMPLES

The following examples are intended only to illustrate methods and embodiments in accordance with the invention and as such should not be construed as imposing limitations upon the claims.

Table 1 lists three strains of Gram negative bacteria, three strains of Gram positive bacteria, two bacterial viruses, and one human virus that were used in the present Examples. The bacterial viruses MS2 and PRD1 were supplied at a concentration of 10⁹ colony-forming units (cfu) per milliter (ml), and the polio virus was at a concentration of 10⁷ plaque-forming units per (pfu)/ml. The viruses were each provided by the Department of Microbiology at the University of Florida.

TABLE 1
Selected microorganisms

| Gram negative | Gram positive | Bacterial virus | Human virus |
|---|---|---|---|
| Escherichia coli | Staphylococcus | MS2, PRD1 | Polio |
| (EC), | aureus (SA), | | |
| Salmonella typi | Enterococcus | | |
| (ST), | faecalis (EC), | | |
| Pseudomonas | Bacillus cereus | | |
| aeruginosa (PA) | (BC) | | |

Bacteria were inoculated into 100 ml of 0.35% Columbia broth, at a concentration of 10⁷ cfu/ml, and were incubated at 37° C., at 200 rotations per minute (rpm). This process was repeated four times in order to completely remove the broth and traces of nutrients. Finally, the bacteria were re-suspended in sterile deionized water to a final concentration of 10⁹ cfu/ml.

Stock solutions of bacteria or viruses were prepared by adding 0.1 ml of test microorganism to 9.9 ml of test medium, resulting in a dispersion having a final concentration of bacteria or virus of 10⁷ cfu/ml or 10⁷ pfu/ml respectively. The experiments were run four hours. Samples were removed at 1 hour and 4 hours and plated in order to count and determine the viable number of microorganism remaining in the sample. The method used for assaying the bacteriophages is detailed in the reference “Influence of Physical and Chemical Treatment on Survival and Association with Flocs under Laboratory Conditions” by S. R. Farrah, P. R. Scheuerman, R. D. Eubanks and G. Bitton, Wat. Sci. Tech. Vol 17, 165-174, 1985, the entire contents of which are hereby incorporated by reference. The method used for assaying the viruses is detailed in the reference “Influence of Slats on Virus Adsorption to Microporous Filters,” J. Lukasil, T. M. Scott, D. Andryshak, S. R. Farrah, Applied and Environmental Microbiology, vol 66, 2914-2920, 2000, the entire contents of which are hereby incorporated by reference.

Each test was carried out in triplicate. The log reduction of the test microorganism was calculated using the Formula I below:

Log reduction=Log(N_{control})−Log(Nₜₑₛₜ). Formula I:

In the above Formula I, N_{control} is the concentration of bacteria present in the control test at 1 hour (PBS, pH=7.4), and Nₜₑₛₜ is the concentration of the bacteria present in the test samples following either 1 hour or 4 hour incubations.

Granular dialdehyde starch (DAS) was purchased from Sigma without further purification. The DAS from Sigma (P9265) is highly oxidized, having a reported level of oxidation of 73% and containing 10% water. The DAS samples, were suspended in water, heated (“cooked') at 95° C. for two hours with stirring, and then cooled down to room temperature (RT). The solubility of the DAS in water prior to heating is extremely low, however, using the preparation methods (i.e., heating) adapted here, gel suspensions were obtained. The pH values of the prepared DAS gel suspensions are shown in Table 2.

TABLE 2
pH values of the DAS before and after heating

| | Before heating | After heating |
|---|---|---|
| DAS | 3.4 | 2.7-3.0 |

Phosphate buffer saline (PBS) solutions were prepared and the pH of the PBS solutions, ranging from about 2.8 to about 8.7, was adjusted using HCl/NaOH.

For the bacterial experiments utilizing sonicated DAS, the DAS samples were sonicated for about 1 hour prior to initiating the experiments unless otherwise noted. For the experiments testing viruses, including MS2, PRD1 and Polio, DAS samples were not sonicated.

### Example 1

### Evaluation of DAS Effects on Bacteria

This example was conducted to demonstrate the antibacterial activity of heated DAS on three different strains of Gram-negative bacteria and three different strains of Gram-positive bacteria. The minimum lethal concentrations (MLC) for the DAS on both the Gram-negative and Gram-positive bacteria are shown in the table 3.

TABLE 3
MLC (weight percentage) of DAS showing a 7 log reduction
in bacteria following a one hour exposure test.

| | EC | ST | PA | SA | EF | BC |
|---|---|---|---|---|---|---|
| DAS(%) | 0.8 | 2.1 | 1 | 0.8 | 1 | 0.2 |
| pH | 3.2 | 2.9 | 3.1 | 3.2 | 3.1 | 3.5 |

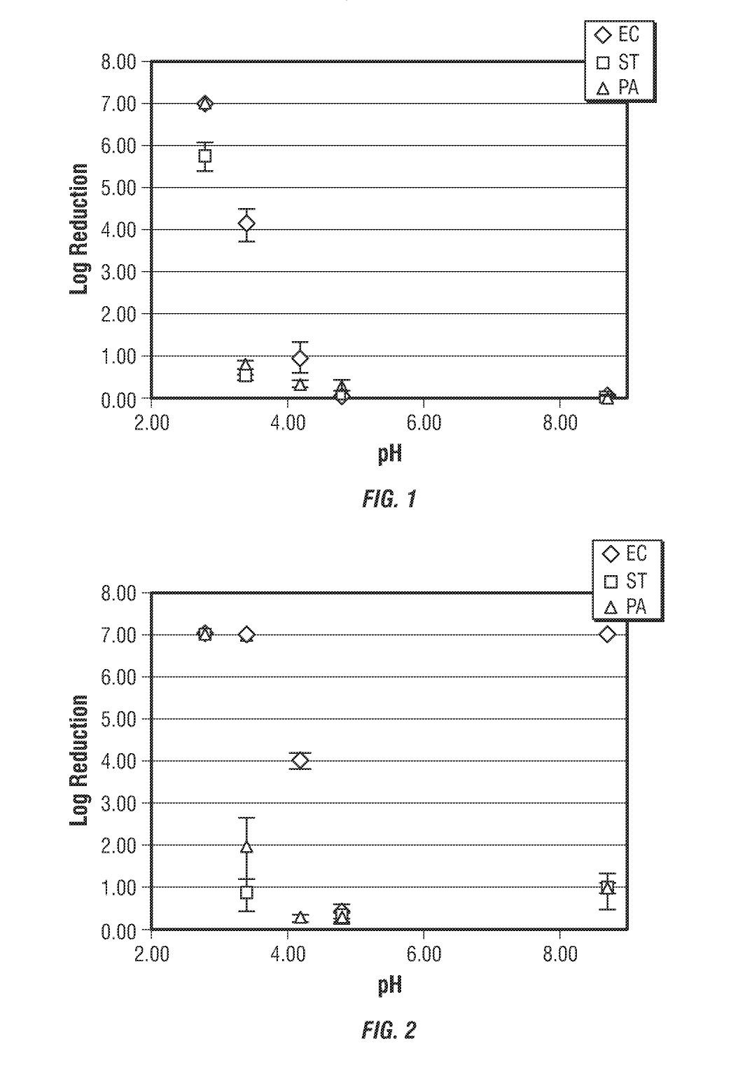

Following the heating or “cook” step, the DAS aqueous suspension was acidic. In order to understand the antimicrobial behavior of DAS against bacteria, that is, whether the acidity or the aldehyde functional groups were contributing to the antimicrobial behavior, additional experiments were conducted. The effect of pH on the log reduction (inactivation) of bacteria was studied using both PBS and DAS, as shown in FIGS. 1-4.

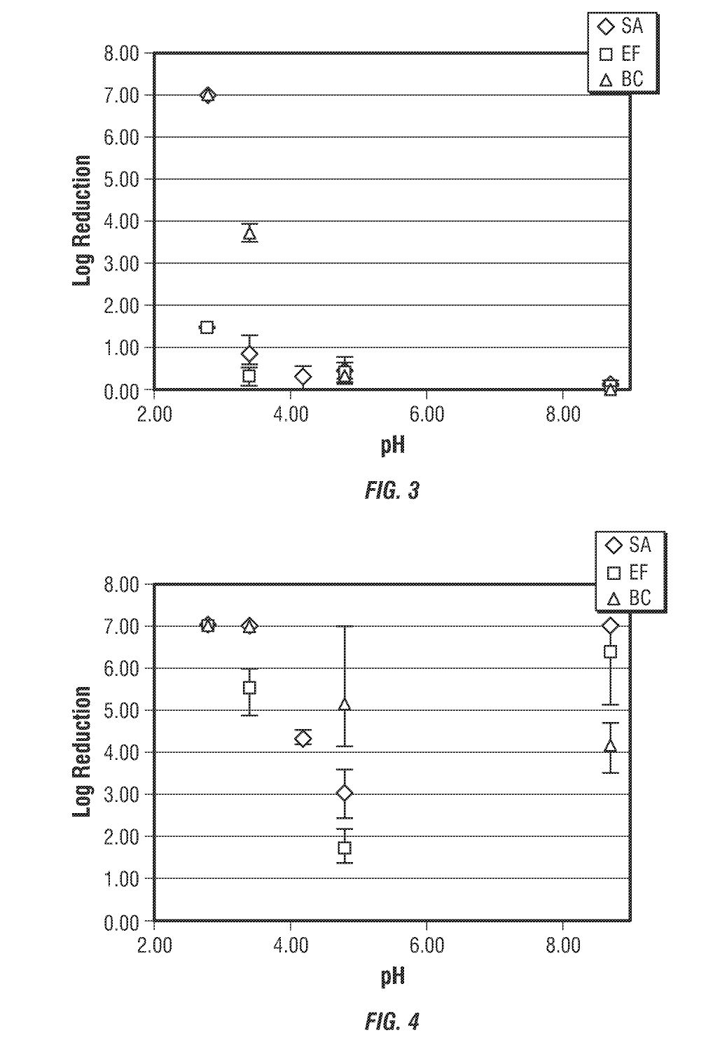

FIGS. 1 and 3 show the effect of the pH of PBS on the log reduction of both Gram-negative and Gram-positive bacteria respectively, while FIGS. 2 and 4 show the effect of the pH of DAS on the log reduction of both Gram-negative and Gram-positive bacteria respectively. As can be seen in the Figures, the effects of pH on the inactivation of bacteria are not the same for PBS as they are for DAS, even though at the low pH value, i.e., around pH=3, PBS can also kill most of the bacteria (with the exception of EF). However, while no inactivation was observed using PBS in the base condition, DAS in the base condition demonstrated strong antimicrobial activities against the three Gram-positive bacterial strains and against one Gram-negative bacterial (EC) strain. Furthermore, the inactivation pH range of DAS was much wider than the pH range of PBS, as demonstrated in all three-Gram positive strains and one Gram-negative strain (EC).

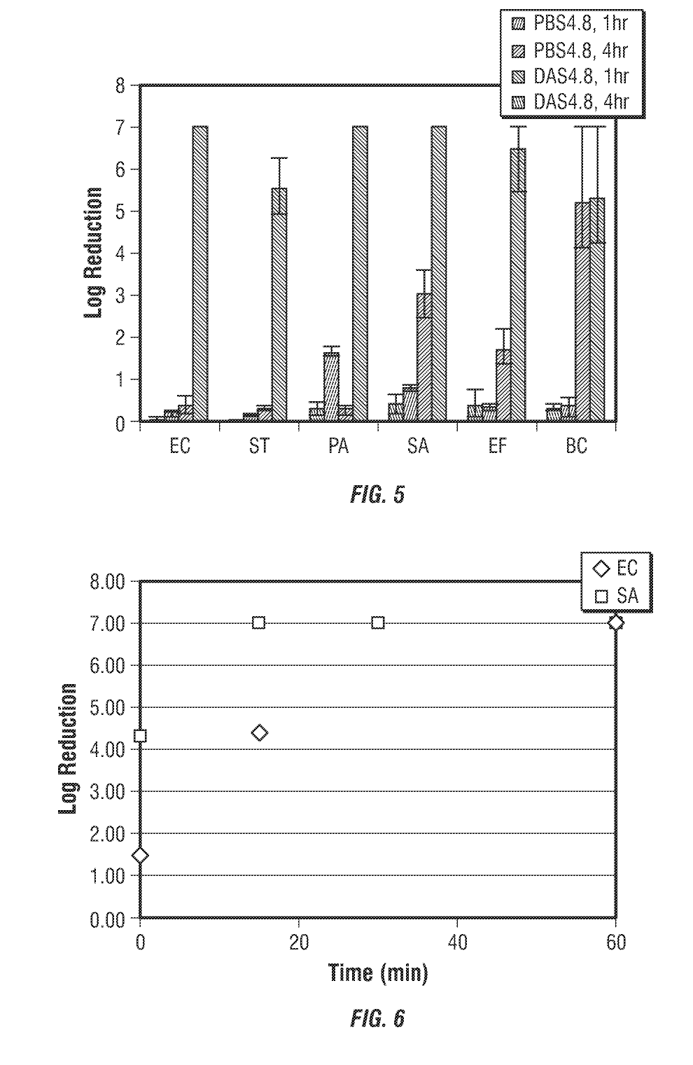

The effect of DAS pH on the inactivation of bacteria may be related to the effect of pH on the activity of the DAS aldehyde groups. Under mild, more basic conditions, it may take a longer period of time for DAS to cause the inactivation of the bacteria. A pH equal to 4.8 was chosen for both PBS and DAS, in order to evaluate the test incubation time on the inactivation of the bacteria. The results for this experiment are shown in FIG. 5. As can be seen in FIG. 5, DAS with a pH=4.8 had no effect on the inactivation of Gram-negative bacteria following incubation for one hour, whereas the antimicrobial activities of DAS against bacteria were significantly increased following an incubation time of four hours. However, there was no time effect associated with the activity of PBS against the bacteria. Based on these results, the aldehyde groups of the DAS play an important role in the inactivation of bacteria.

### Example 2

### Effect of DAS Sonication on Bacterial Inactivation

This example was conducted to show the antibacterial activity of sonicated

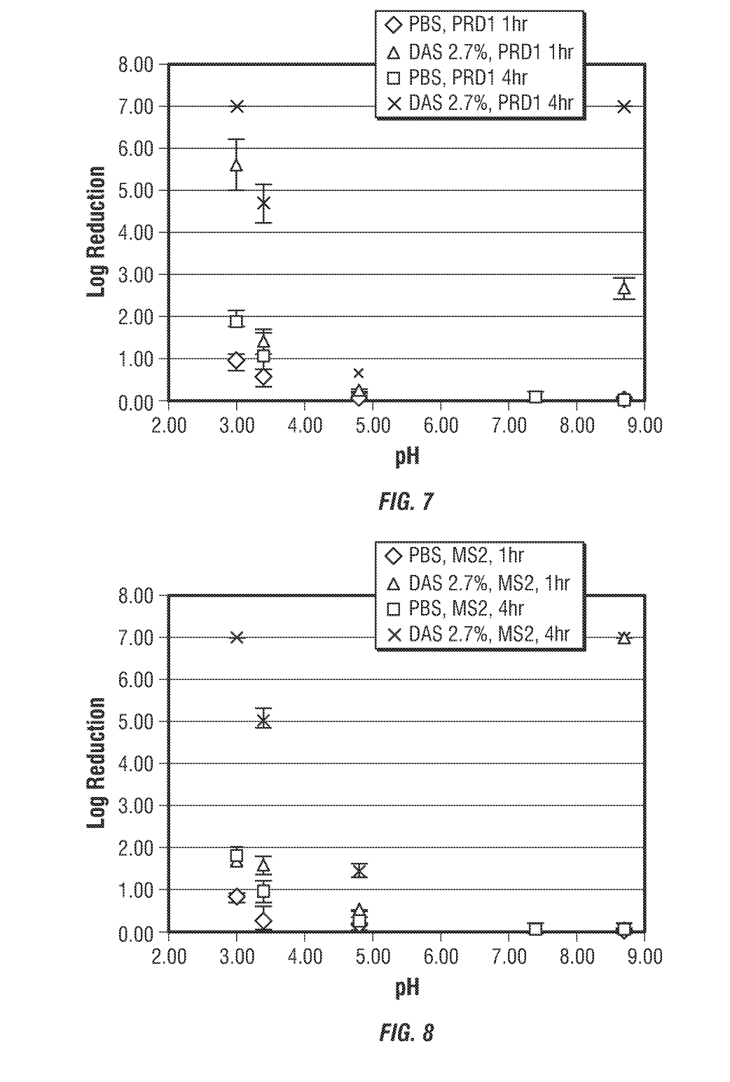

DAS on both Gram-negative and Gram-positive bacterial strains. DAS samples (2.7 wt %) were sonicated for different periods of time, and the effect of sonication on DAS antibacterial activity was evaluated. The pH values of 2.7% DAS at the different sonication times were almost the same, i.e., a pH of about 3. The results in FIG. 7 show the effects of DAS sonication time on bacterial log reduction. As shown in FIG. 7, the antimicrobial activity of DAS against both EC (Gram negative) and SA (Gram positive), was significantly improved by sonicating the DAS for at least 15 minutes. These results are another indication that the antimicrobial activity of DAS is attributable to the activity of the aldehyde groups. The particle size of DAS in the sonicated suspension was in the micrometer range. Once the DAS gel was broken down by the sonication, more aldehydes groups could be exposed to the bacterial, even though the pH values remained almost the same. The antimicrobial activity of DAS following sonication was significantly improved, as seen in FIG. 7. After 30 minutes of sonication, DAS completely kills the bacteria. As a comparison, while the non-sonicated samples were unable completely inactivate all of the bacteria in one hour, they were able to completely kill (inactivate) the bacteria in a four hour period (data not shown).

### Example 3

### Evaluation of DAS Effects on Bacterial Viruses

This example was conducted to show the antiviral activity of DAS on bacterial viruses. For the bacteriophage experiments, the heated DAS samples were employed. The antiviral activities of DAS against two bacterial viruses, PRD1 and MS2, are presented in FIGS. 7 and 8. Unlike the effect of pH on the antibacterial activity of PBS, no effect of pH on the antiviral activity of PBS, against these two bacterial viruses, were observed. In both the low pH (pH=3) and high pH (pH=8.7) samples, DAS was able to completely kill all of the PRD1 and MS2 bacteriophage, during an incubation period of 4 hours. However, as shown in FIG. 8, at a pH=8.7, DAS can also completely kill the MS2 bacteriophage within a period of only one hour. As shown in FIG. 7, a lower level of activity against PRD1 was observed. At a pH=3, DAS has a higher level of activity against PRD1 than against MS2 in the one hour test. As compared to the antibacterial results, the activity of DAS against the bacterial viruses follows the same trend as observed with the bacterial. That is, at some pH values (i.e. pH=4.8), the antiviral activity of DAS was not observed in one hour experiments. Further, when the test time was increased to 4 hours, no significant improvement in antiviral activity could be observed.

The effects of one-hour sonication of DAS samples against bacterial viruses were not observed (data not shown here), probably because the size of the viruses are much smaller than the bacteria, and the number of aldehyde groups exposed on the DAS is sufficient to kill the viruses without having to expose additional aldehyde groups.

### Example 4

### Human Polio Virus

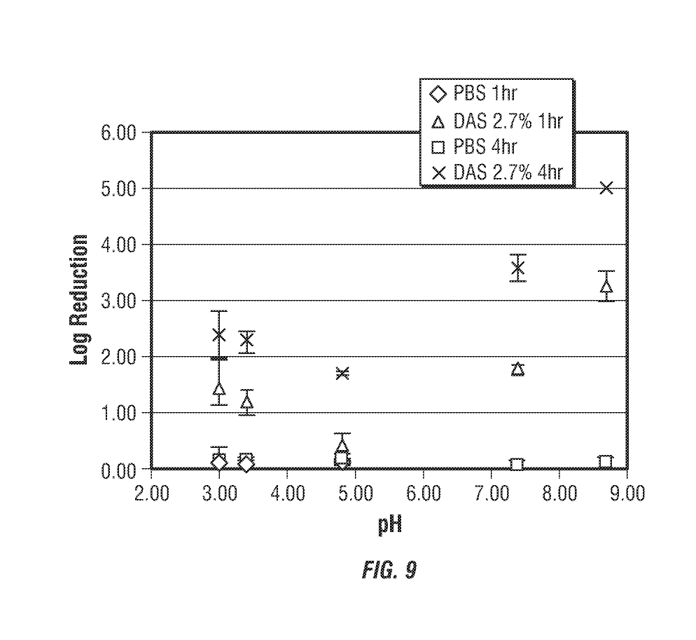

This example was conducted to examine the antiviral activity of heated DAS on the human Polio virus. The results of heated DAS activity on the Polio virus are shown in FIG. 9. As was shown for the bacterial MS2 and PRD1 viruses, PBS had no observable effect on the viability of the Polio virus, regardless of the pH. As shown in FIG. 9, the antiviral activity of DAS against the Polio virus, at a pH=8.7, is significantly stronger than the antiviral activity observed under acidic conditions.

Thus in summary, DAS which has been heated for 2 h at a temperature of 95° C., is able to effectively inactivate both Gram-negative and Gram-positive bacteria as well as both RNA (polio, MS2) and DNA viruses (PRD1) within a period of 4 hours. Further, sonicated DAS is also able to effectively kill both Gram-negative and Gram-positive bacteria within a period of 4 hours.

### Example 5

This example was conducted to determine the properties of the antimicrobial agent that is produced after heating of the dialdehyde starch in water or after the heating of oxidized corn starch in water. The water was deionized prior to heating with either of the starches.

The dialdehyde starch heated in water was characterized using gel permeation chromatography to determine the molecular weight. Fourier Transform Infrared Spectrometry and Ultraviolet Visible Spectroscopy were used to determine the structure of the antimicrobial agent.

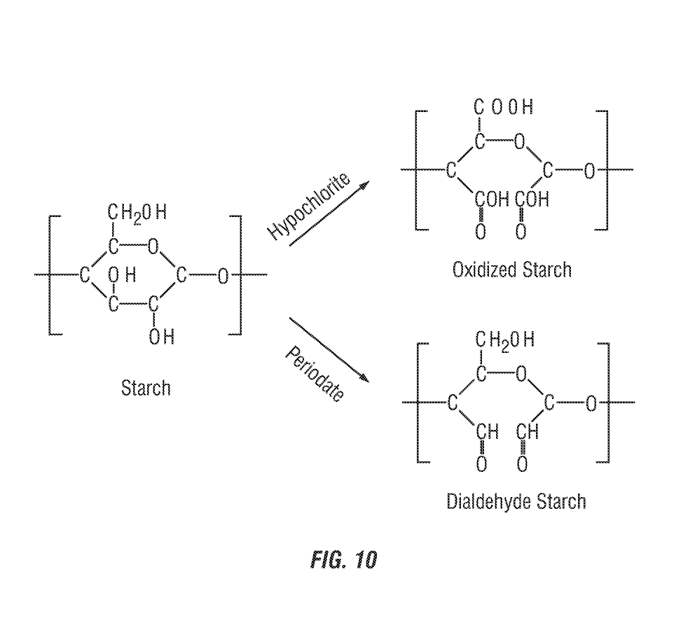

The dialdehyde starch was obtained from Sigma (P9265), while the oxidized corn starch was obtained from Grain Processing Corporation, Muscatine, Iowa, USA. Both starches were used without further purification. The difference between the structures of the dialdehyde starch and the oxidized corn starch are shown in the FIG. 10. Three grams of the dialdehyde starch were heated in deionized water for two hours each at 90° C. to 95° C.

Five weight percent of the oxidized starch was heated in deionized water in the same manner. The weight percents were based on the total amount of the oxidized starch and the deionized water. The heating was conducted in an oil bath and the deionized water was refluxed during the heating. The suspension manufactured as a result of the heating was cooled to room temperature. The pH values of the 3% starch suspensions before the cook and after the cook were ca. 3.8 and 3 respectively.

The solubility of the dialdehyde starch in deionized water was determined by the combination of centrifugation and freeze-drying. Upon dissolution in deionized water, the sample is referred to as the “as-prepared dialdehyde starch aqueous suspension”. The dialdehyde granular suspensions were centrifuged at 10,000 g RCF (relative centrifugal force) at 4° C. for 30 minutes to obtain the dialdehyde starch sedimentation fraction and the supernatants. The dialdehyde starch sedimentation and the supernatants were freeze-dried for 24 hours, and the solid content in each fraction was determined The solubility was expressed as the weight percentage of the solid in the dialdehyde starch supernatant over total solid weight of dialdehyde starch.

### Gel Permeation Chromatography Characterization

Molecular weight of the as-received dialdehyde starch and freeze-dried dialdehyde starch from the supernatant of the as-prepared dialdehyde starch aqueous suspension was determined by a gel permeation chromatography (GPC) (PL-GPC, Polymer Laboratories, Amherst, Mass.) with a differential refractive index detector and three phynogel columns The mobile phase was dimethyl sulfoxide (DMSO) containing 5 millimolar (mM) NaNO₃ at a flow-rate of 0.8 milliliters per minute (ml/min). The columns were calibrated with a series of dextran narrow standards (American Polymer Standards Corporation, Mentor, Ohio).

DAS sample (8 milligrams (mg)) was dissolved in DMSO (4 ml) by heating for 120 minutes in a boiling water bath and the solution was filtrated through a 2.0 μm filter prior to the GPC analysis.

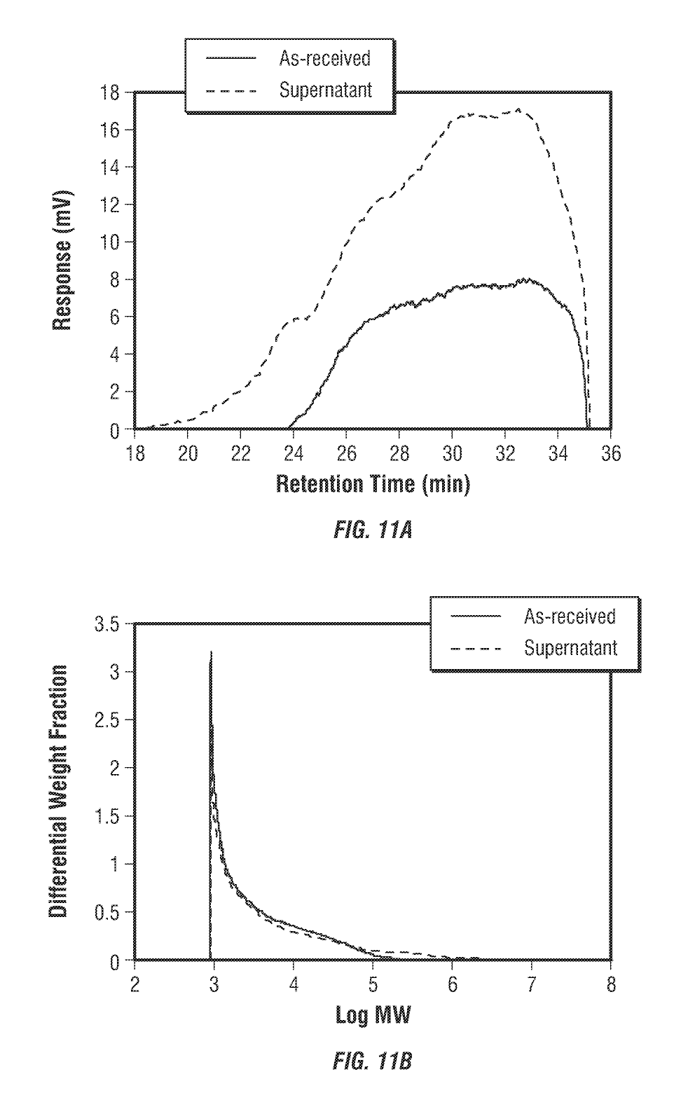

The results are shown in the FIG. 11(a) and (b) respectively. FIG. 11(a) and (b) are gel permeation chromatography analysis of the as-received dialdehyde starch and the dialdehyde starch solids from the supernatant. FIG. 11(a) is a graph showing the response versus the retention time, while the FIG. 11(b) is a graph showing the differential weight fraction versus molecular weight.

In general, the molecular weight and distribution pattern were similar for the as-received dialdehyde starch as for the supernatant of the as-prepared dialdehyde starch aqueous suspension. Both samples displayed a peak value of molecular weight around 900 Daltons. The lack of a small weight percentage of the high molecular weight portion in the as-received dialdehyde starch was probably due to the fact that the as-received dialdehyde starch was observed not to be dispersed completely during the gel permeation chromatography sample preparation. Gel permeation chromatography analysis also suggested that the change of solubility and particle size of the as-prepared dialdehyde starch aqueous suspension would be mainly caused by the physical disruption of the dialdehyde starch granules during the cooking.

### Fourier Transform Infrared Spectrometry (FTIR) Characterization

The infrared spectra of the dialdehyde starch solids (1% in KBr) were obtained by a Thermo electron magna 760 FTIR with a DTGS detector in a diffuse reflection mode using 128 scans at resolution of 4 cm⁻¹.

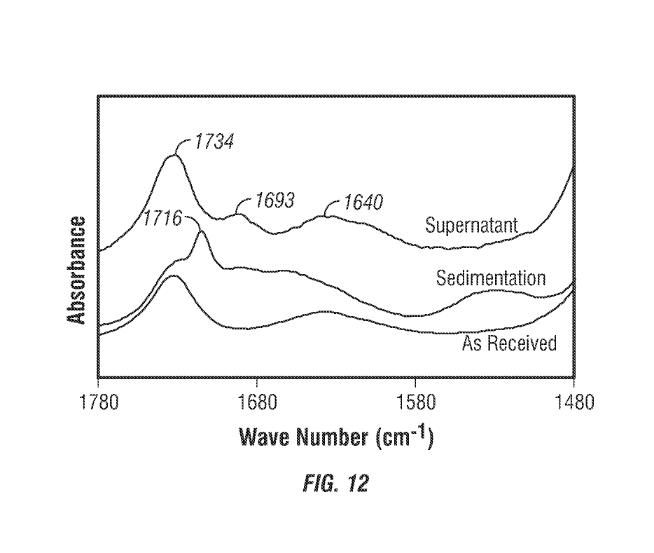

Even though the degradation of the dialdehyde starch during cooking was found to be limited, as the color of the dialdehyde starch aqueous suspension changed to yellow, chemical changes may have taken place as indicated in FIG. 12. The FIG. 12 shows the FTIR spectra of the as-received dialdehyde starch granule, a freeze dried sample of the as-prepared 3% dialdehyde starch aqueous supernatant and a sample comprising the sedimentation after centrifugation.

All of the dialdehyde starch samples had a characteristics absorption band around 1734 cm⁻¹ in the FTIR spectra, revealing the stretching vibration of the carbonyl group. There was a new band around 1693 cm⁻¹ and 1716 cm⁻¹ for the dialdehyde starch aqueous suspension supernatant and sedimentation respectively. In the study of synthesis and characterization of polyglutaraldehyde, the FTIR spectra of polyglutaradehyde showed bands at 1720 and 1680 cm⁻¹. The 1720 cm⁻¹ absorbance peak was assigned to the nonconjugated aldehyde or carboxylic acid and the 1680 cm⁻¹ was assigned to the conjugated aldehyde. It is difficult to distinguish the C═C bond and the OH of water, since both are at the same wavelength 1640 cm⁻¹. Without being limited to theory, it is proposed that the 1734 cm⁻¹, 1693 cm⁻¹ and 1716 cm⁻¹ wavelength bands in the dialdehyde starch samples are the stretching of the C═O of the non-conjugated aldehyde, the stretching of the C═O of the conjugated aldehyde and the stretching of the C═O of the carboxylic acid respectively. The formation of the conjugated aldehdye and carboxylic acid of the dialdehyde starch aqueous suspension may be formed by the β-elimination and Cannizzaro reaction respectively. The formation of the conjugated aldehyde would explain the color change to yellow of the dialdehyde starch aqueous suspension and the formation of the carboxylic acid would explain the pH decrease of the dialdehyde starch aqueous suspension during the cooking.

### Ultraviolet-Visible (UV-Vis) Spectroscopy Characterization

The UV-Vis spectra of the dialdehyde starch suspensions and the dialdehyde starch solids were obtained with a Perkin-Elmer Lambda 800 UV-VIS spectrometer in the transmission and reflectance modes respectively. Quartz cuvettes were used for the suspension measurement.

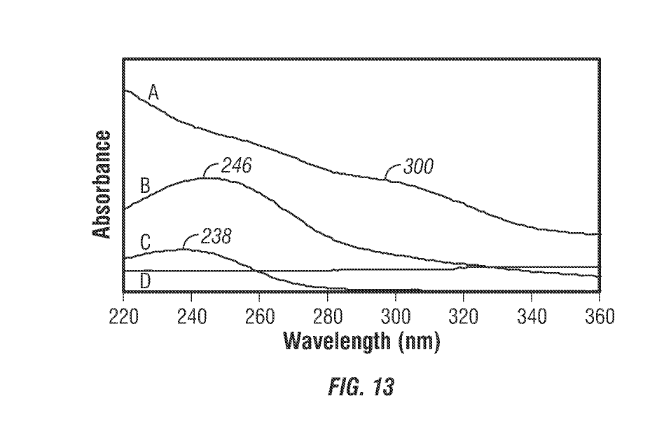

The formation of the conjugated aldehyde was further confirmed by the UV-Vis spectra shown in the FIG. 13. FIG. 13(a) is a UV-Vis spectra of the dialdehyde starch samples taken in the reflectance mode for as-received dialdehyde starch. FIG. 13(b) is a UV-Vis spectra of the freeze-dried sample of the 3% as-prepared dialdehyde starch aqueous supernatant taken in the reflectance mode. FIG. 13(c) is a UV-Vis spectra taken in the transmission mode of the 3% as-prepared dialdehyde starch aqueous supernatant at pH=3, diluted 100 times using same pH PBS buffer. FIG. 13(d) is a UV-Vis spectra taken in the transmission mode of the 0.3% dialdehyde starch granular suspension.

For the suspensions, no absorbance peaks were observed for the dialdehyde starch granular suspension. This result was probably caused by the sedimentation of the dialdehyde starch granules.

A strong absorbance peak at 238 nanometer (nm) wavelength was exhibited in the 0.03% dialdehyde starch aqueous suspension supernatant. A strong peak at 246 nm and a weak peak at 300 nm were observed in the reflectance mode of the freeze-dried 3% dialdehyde starch aqueous suspension supernatant and as-received dialdehyde starch granule respectively. The commercial glutaradehyde suspension exhibits two absorption maxima in the range of 225 to 245 nm and 270 to 290 nm, commonly at 235 nm and 280 nm.

After carefully purification, the absorption at 235 nm can be eliminated. Reported studies indicate that the monoglutaraldehyde (non-conjugated aldehdye) and polymeric glutaraldehyde (conjugated aldehdye) are responsible for the absorption at 280 and 235 nm respectively. The 238 and 246 nm absorbance of the 3% dialdehyde starch aqueous supernatant in the transmission and reflectance modes supported our FTIR studies that a conjugated aldehyde function with an ethylenic linkage (C═C—C═O) was formed. The absorption of the non-conjugated aldehyde in the range of 270 to 290 nm was not detected by the UV-Vis, even though it was confirmed by the FTIR. It was probably caused by the high extinction coefficient (ε=18.6 L g⁻¹ cm⁻¹) of the conjugated aldehyde compared to the non-conjugated aldehyde (ε=4.2×10⁻² L g⁻¹ cm⁻¹). A weak absorption at 300 nm was observed for the as-received dialdehyde starch granules in the reflectance mode. This absorption could be the non-conjugated aldehyde function. The conjugated aldehyde absorption was not observed in the dialdehyde starch granule case, as it should be much stronger than the non-conjugated aldehyde absorption. The FTIR and UV-Vis spectra clearly demonstrated that the conjugated aldehyde was formed during the cooking, probably caused by the β-elimination.

### Example 6

This example was conducted to demonstrate the efficacy of a filter containing the dialdehyde polysaccharides disclosed above. Sodium periodate (NaIO₄) and Whatman 50 plain cellulose filter paper (particle retention >2.7 mm, 5.5 cm diameter) were purchased from Fisher Scientific. Twelve pieces of cellulose paper (total weight ca. 2.72 grams) was immersed into 100 ml deionized water containing 0.2 molar (M) sodium periodate. The pH of the periodate solution was 3 to 4. The reaction was kept dark at 37° C. in a shaker for a certain time. The speed of the shaker was 200 revolutions per minute. After the reaction, filter papers were first washed five times with deionized water. These filter papers were immersed into 100 ml deionized water in a shaker at room temperature overnight. They were washed five times again with deionized water. 2 ml 0.5% (w/v) sodium metabisulfite aqueous solution was spread onto a filter paper, no color change was observed. This indicated no residue periodate. Portions of the cellulose filter are converted to dialdehyde polysaccharide. After completely rinse of residue periodate, they were dried in a hood at room temperature for 24 hours.

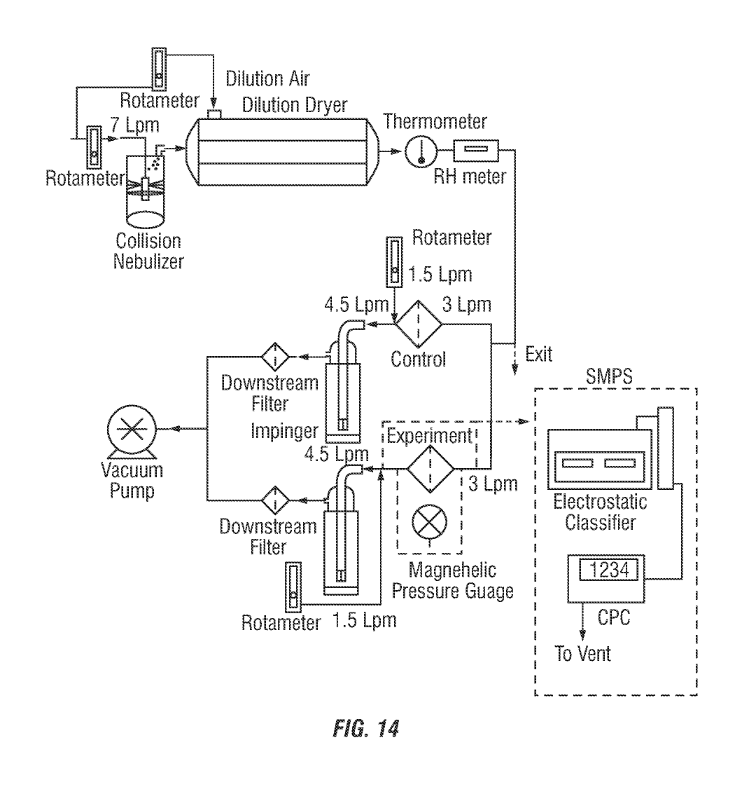

The experimental setup is shown in the FIG. 14. As demonstrated in FIG. 14, the experimental set-up had two components. In one set-up, the control had no filter, while the alternative setup was conducted with a filter. Pressure drop, physical removal efficiency (PRE), viable removal efficiency (VRE) and infectivity of virus on the filter were obtained. PRE was determined as shown in the Equation (II) below:

$$PRE ( \% ) = ( 1 - \frac{{N}_{p}}{{N}_{E}} ) \times 100 \qquad ( II )$$

where N_{E} is the number of particles entering the filter and Nₚ is the number of particles penetrating the filter. VRE was determined by counting plaques of virus collected from control and experimental impingers as determined by the Equation (III).

$$VRE ( \% ) = ( 1 - \frac{{C}_{ctr}}{{C}_{test}} ) \times 100 \qquad ( III )$$

where C_{ctr} is the number of virus collected from the control impinger and Cₜₑₛₜ is the number of virus collected from the experimental impinger.

Infectivity of virus on the filter paper was determined by counting plaques of virus recovered from untreated and treated filter paper. The extracted fraction is defined as the ratio of the infectivity count in the extract solution to the total viruses collected on the filter.

Experiments were conducted at room temperature and two relative humidities (RH): medium RH (55±5%, MRH) and high TH (90±5%, HRH). Experiments were conducted in triplicates in each environmental condition. The results are shown in the Tables 4 and 5 below.

TABLE 4

| | | Pressure drop | | | | Quality |
|---|---|---|---|---|---|---|
| Environmental | | at 5.3 cm/s | | | Extracted | Factor* |
| Condition | Test Filter | (in. H₂O) | PRE (%) | VRE* (%) | Fraction* | (kPa⁻¹) |
| RT/LRH | Untreated | 15.8^{#} | 99.9999 | 82.1 ± 2.6 | 0.43 ± 0.16 | 0.44 ± 0.04 |
| | 12-hr treated | 6.5 | 92.0663 | 78.3 ± 4.9 | 0.94 ± 0.21 | 0.24 ± 0.16 |
| RT/MRH | Untreated | 15.8^{#} | NA^{†} | 72.1 ± 2.6 | 0.51 ± 0.03 | 0.32 ± 0.02 |
| | 12-hr treated | 6.5 | NA | 75.5 ± 8.9 | 0.28 ± 0.12 | 0.87 ± 0.28 |
| RT/HRH | Untreated | 15.8^{#} | NA | 79.8 ± 1.7 | 0.43 ± 0.20 | 0.41 ± 0.02 |
| | 12-hr treated | 6.5 | NA | 89.2 ± 3.7 | 0.19 ± 0.01 | 1.37 ± 0.26 |

*The average measurements in triplicate,
^{†}Not available,
^{#}Not measured

TABLE 5

| | | Pressure drop | | | | Quality |
|---|---|---|---|---|---|---|
| Environmental | | at 5.3 cm/s | | | Extracted | Factor* |
| Condition | Test Filter | (in. H₂O) | PRE (%) | VRE* (%) | Fraction* | (kPa⁻¹) |
| RT/LRH | Untreated | 15.8 | 87.8703 | 72.1 ± 5.3 | 0.74 ± 0.04 | 0.32 ± 0.05 |
| | 12-hr treated | 6.2 | 78.5355 | 70.3 ± 2.4 | 0.73 ± 0.07 | 0.78 ± 0.02 |
| RT/MRH | Untreated | 15.8 | 88.2918 | 83.1 ± 9.3 | 0.54 ± 0.24 | 0.45 ± 0.20 |
| | 12-hr treated | 6.2 | 77.8234 | 90.3 ± 2.4 | 0.47 ± 0.11 | 1.51 ± 0.11 |
| RT/HRH | Untreated | 15.8 | 88.1872 | 88.1 ± 3.3 | 0.58 ± 0.01 | 0.54 ± 0.08 |
| | 12-hr treated | 6.8 | 80.8232 | 93.3 ± 6.7 | 0.22 ± 0.08 | 1.59 ± 0.40 |

From the Tables 4 and 5, it can be seen that the dialdehyde starch cellulose process makes the pores larger (as expressed by lower pressure drop, 40%). So, the physical efficiency (PRE) of the treated filter is not as good as the untreated filter.

At higher humidity, the filter works better than at lower relative humidity. This is show in viable removal efficiency (VRE) and extracted fraction. Viable removal efficiency is defined as 100%—viable count downstream the filter/viable count upstream the filter (the higher the better). Extracted fraction is defined as viable count extracted from the filter/viable count collected on the filter (the lower, the better).

Better performance at higher humidity is also expressed by the higher quality factor, which is defined as log(penetration)/pressure drop. The higher, the better.

A lower extracted fraction indicates that the collected micro-organisms on the filter are inactivated. Higher filter quality indicates that some microbes are inactivated while flying through the filter, i.e., direct contact with the filter surface is not absolutely required.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention.

1. A method of preparing an antimicrobial agent, comprising:
heating a dialdehyde polysaccharide in water at a temperature of about 60° C. to about 120° C. to form a dispersion of dialdehyde polysaccharide in water.

2. The method of claim 1, wherein the heating is conducted for a period of about 1 to about 4 hours.

3. The method of claim 1, wherein the heating is conducted for a period of about 1.5 to about 3 hours.

4. The method of claim 1 further comprising sonicating the dispersion of dialdehyde polysaccharide in water.

5. The method of claim 4, wherein the dispersion of dialdehyde polysaccharide in water is sonicated for about 15 minutes to about 90 minutes.

6. The method of claim 5, wherein the dispersion of dialdehyde polysaccharide in water is sonicated for about 30 to about 60 minutes.

7. A device that employs the method of claim 1.

8. An article manufactured by the method of claim 1

9. The article of claim 9, wherein the article comprises gloves, masks, clothing, garments, tampons, incontinence pads, sheets, surgical or burn dressings, adhesive bandages, balloons or catheter tips adapted for insertion into a living being, prosthetic heart valves, sutures, surgical staples, synthetic vessel grafts, stents, stent grafts, vascular or non-vascular grafts, shunts, aneurysm fillers, intraluminal paving systems, guide wires, embolic agents, filters, drug pumps, arteriovenous shunts, artificial heart valves, artificial implants, foreign bodies introduced surgically into the blood vessels or at vascular or non-vascular sites, leads, pacemakers, implantable pulse generators, implantable cardiac defibrillators, cardioverter defibrillators, defibrillators, spinal stimulators, brain stimulators, sacral nerve stimulators, chemical sensors, breast implants, interventional cardiology devices, catheters, plastic tubing, catheters, dialysis bags or membranes whose surfaces come in contact with the blood stream of a patient.

10. An antimicrobial composition, comprising:
a dispersion of a dialdehyde polysaccharide in water; the dispersion having a pH of about 2.5 to about 9.

11. The composition of claim 10, wherein an amount of dialdehyde polysaccharide ranges from about 0.5 to about 10 weight percent, based upon the total weight of the antimicrobial composition.

12. The antimicrobial composition of claim 10, further comprising an additives selected from the group consisting of pigments, fragrances, anticorrosion agents, stabilizers, surfactants, and a combination comprising at least one of the foregoing additives.

13. The antimicrobial composition of claim 10, wherein the dialdehyde polysaccharide is selected from a dialdehyde starch or a dialdehyde cellulose.

14. A method of inhibiting the growth of a microbial agent, the method comprising:
contacting the microbial agent with a composition comprising a dispersion of dialdehyde polysaccharide in water; the dispersion having a pH of about 2.5 to about 9.

15. The method of claim 14, wherein the dialdehyde polysaccharide is selected from a dialdehyde starch or a dialdehyde cellulose.

16. The method of claim 14, wherein the microbial agent is selected from the group consisting of a Gram-negative bacteria, a Gram-positive bacteria, a virus, and a combination comprising at least one of the foregoing microbial agents.

17. The method of claim 14, wherein the contacting of the microbial agent with the composition comprising a dialdehyde polysaccharide is for a period of about 0.5 to about 10 hours.

18. A method of producing an antimicrobial article comprising:
heating a dialdehyde polysaccharide in water at a temperature of about 60° C. to about 120° C. to form a dispersion of dialdehyde polysaccharide in water;
sonicating the dispersion of dialdehyde polysaccharide in water; and
contacting the article with the dispersion of dialdehyde polysaccharide in water to form an antimicrobial article.

19. A composition comprising:
a dialdehyde polysaccharide; and
water; the dialdehyde polysaccharide being dispersed in the water; the dialdehyde polysaccharides having average particle sizes of about 5 to about 150 nanometers.

20. The composition of claim 19, wherein the dialdehyde polysaccharides are a dialdehyde starch, a dialdehyde cellulose, cellulose, alkyl cellulose, methyl cellulose, hydroxyalkyl cellulose, alkylhydroxyalkyl cellulose, cellulose sulfate, salts of carboxymethyl cellulose, carboxymethyl cellulose, carboxyethyl cellulose, chitin, carboxymethyl chitin, hyaluronic acid, salts of hyaluronic acid, alginate, alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, chitosan, heparin, heparin sulfate, heparin sulfate, dermatan sulfate, keratin sulfate, carrageenans, chitosan, starch, amylose, amylopectin, poly-N-glucosamine, polymannuronic acid, polyglucuronic acid, polyguluronic acid, and derivatives of any of the above, or a combination comprising one or more of the foregoing dialdehyde polysaccharides.

21. The composition of claim 19, where the composition has a viscosity of about 0.03 to about 0.3 poise when measured at room temperature.

22. The composition of claim 19, where the dialdehyde polysaccharide is present in an amount of about 1 to about 99 weight percent, based on the total weight of the composition.

23. An article comprising the composition of claim 19.

24. The composition of claim 19 being in the form of an aerosol.

25. A filter comprising:
a substrate; and
a dialdehyde polysaccharide disposed upon the substrate.

26. The filter of claim 25, where the substrate is porous.

27. The filter of claim 25, wherein the dialdehyde polysaccharides are a dialdehyde starch, a dialdehyde cellulose, cellulose, alkyl cellulose, methyl cellulose, hydroxyalkyl cellulose, alkylhydroxyalkyl cellulose, cellulose sulfate, salts of carboxymethyl cellulose, carboxymethyl cellulose, carboxyethyl cellulose, chitin, carboxymethyl chitin, hyaluronic acid, salts of hyaluronic acid, alginate, alginic acid, propylene glycol alginate, glycogen, dextran, dextran sulfate, curdlan, pectin, pullulan, xanthan, chondroitin, chondroitin sulfates, carboxymethyl dextran, carboxymethyl chitosan, chitosan, heparin, heparin sulfate, heparin sulfate, dermatan sulfate, keratin sulfate, carrageenans, chitosan, starch, amylose, amylopectin, poly-N-glucosamine, polymannuronic acid, polyglucuronic acid, polyguluronic acid, and derivatives of any of the above, or a combination comprising one or more of the foregoing dialdehyde polysaccharides.

28. The filter of claim 25, where the filter is a weave, a foam, a textile, a mesh or a gauze.

29. The filter of claim 25, where the substrate comprises cellulose and wherein the dialdehyde polysaccharide is covalently bonded to the cellulose.

30. A method comprising:
oxidizing a cellulose;
forming a dialdehyde polysaccharide on a surface of the cellulose; and
using the cellulose having the dialdehyde polysaccharide disposed thereon as a filter.
